(19) United States
(12) Patent Application Publication
(10) Pub. No.: US 2026/0242361 A1
(43) Pub. Date: Aug. 20, 2026

(54) COMPOSITIONS AND METHODS OF MAKING THE COMPOSITIONS

(71) Applicant: University of Florida Research Foundation, Inc., Gainesville, FL (US)

(72) Inventors: Alexander James Grenning, Gainesville, FL (US); Vincent Vedovato, Gainesville, FL (US)

(21) Appl. No.: 18/992,676

(22) PCT Filed: Aug. 23, 2023
(86) PCT No.: PCT/US2023/072699

§ 371 Date: Jan. 9, 2025

Related U.S. Application Data
(60) Provisional application No. 63/400,159, filed on Aug. 23, 2022.
(60) Provisional application No. 63/472,651, filed on Jun. 13, 2023.

(51) Int. Cl. C07D 405/04; C07D 311/12; C07D 409/04

(52) U.S. Cl. CPC C07D 405/04; C07D 311/12; C07D 409/04

(57) ABSTRACT

The present disclosure provides for polyfunctionalized biaryls having complex biaryl architectures, methods of making poly-functionalized biaryls, and the like. The present disclosure provides for the cine-ipso-difunctionalization transformation of nitrocoumarins. The reaction leverages the electrophilicity of nitrocoumarins, the nucle-ophilicity of nitronates, and the leaving group ability of nitrite (NO₂—) to yield complex, polyfunctionalized biaryls that often display stable axial chirality.

### CLAIM OF PRIORITY TO RELATED APPLICATION

This application claims priority to co-pending U.S. provisional application entitled “COMPOSITIONS AND METHODS OF MAKING THE COMPOSITIONS” having Ser. No. 63/400,159, filed on Aug. 23, 2022, which is entirely incorporated herein by reference. In addition, this application claims priority to co-pending U.S. provisional application entitled “COMPOSITIONS AND METHODS OF MAKING THE COMPOSITIONS” having Ser. No. 63/472,651, filed on Jun. 13, 2023, which is entirely incorporated herein by reference.

### FEDERAL FUNDING

This invention was made with government support under Grant No. 1844443, awarded by the National Science Foundation. The government has certain rights in the invention.

### BACKGROUND

The linking of functional groups and (hetero)aromatics to (hetero)arene building blocks controllably and rapidly is critical considering the prominence of this pattern in pharmaceuticals. Thus, there is a need for further advances to be made involving this chemistry.

### SUMMARY

The present disclosure provides for polyfunctionalized biaryls having complex biaryl architectures, methods of making polyfunctionalized biaryls, and the like.

In an aspect, the present disclosure provides for a composition, comprising a chemical having the following formula:

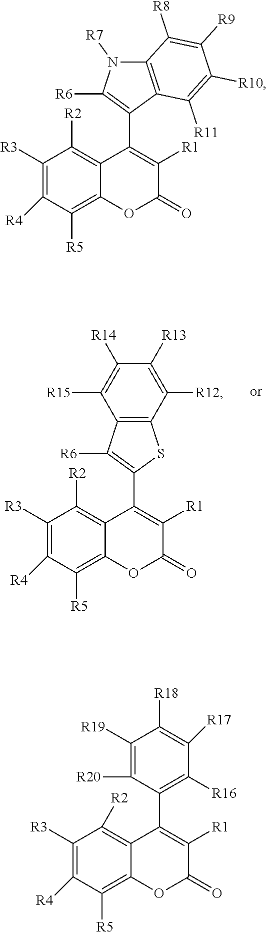

wherein R1 is an alkenyl group or an alkenyl group terminated by an acetoxy (OAc) group or a phenyl group; wherein R2-R5, R8, R9, and R11-R20 are each independently selected from hydrogen, a halogen group, —OH, —CN, —C═N—S(O)(alkyl group), alkynyl group, or —O-alkyl; wherein R6 is selected from hydrogen, a halogen group, —OH, —CN, —C═N—S(O)(alkyl group), —O-alkyl, an alkyl group, a phenyl group, —C(O)O-alkyl, —C(O)H, —C(O)R30, —C(O)NH(alkenyl), or —COHNR31, and wherein R30 is a morpholine group and wherein R31 is hydrogen, a halogen group, or an alkyl group; wherein R7 is H or an alkyl group; and wherein R10 is H, a halogen group, —CHO, —B(OH)₂, or —O(alkyl).

In an aspect, the present disclosure provides for a composition, comprising a chemical having the following formula:

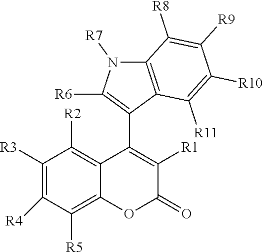

wherein R1 is an alkenyl group or an alkenyl group terminated by an acetoxy (OAc) group or a phenyl group; wherein R2-R5, R8, R9, and R11 are each independently selected from hydrogen, a halogen group, —OH, —CN, —C═N—S(O)(alkyl group), alkynyl group, or —O— alkyl; wherein R6 is selected from hydrogen, a halogen group, —OH, —CN, —C═N—S(O)(alkyl group), —O-alkyl, an alkyl group, a phenyl group, —C(O)O-alkyl, —C(O)H, —C(O)R30, —C(O)NH(alkenyl), or —COHNR31, and wherein R30 is a morpholine group and wherein R31 is hydrogen, a halogen group, or an alkyl group; wherein R7 is H or an alkyl group; and wherein R10 is H, a halogen group, —CHO, —B(OH)₂, or —O(alkyl).

In an aspect, the present disclosure provides for a composition, comprising a chemical having the following formula:

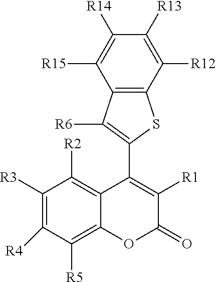

wherein R1 is an alkenyl group or an alkenyl group terminated by an acetoxy (OAc) group or a phenyl group; wherein R2-R5, and R12-R15 are each independently selected from hydrogen, a halogen group, —OH, —CN, —C═N—S(O)(alkyl group), alkynyl group, or —O-alkyl; wherein R6 is selected from hydrogen, a halogen group, —OH, —CN, —C═N—S(O)(alkyl group), —O-alkyl, an alkyl group, a phenyl group, —C(O)O-alkyl, —C(O)H, —C(O)R30, —C(O)NH(alkenyl), or —COHNR31, and wherein R30 is a morpholine group and wherein R31 is hydrogen, a halogen group, or an alkyl group; wherein R7 is H or an alkyl group.

In an aspect, the present disclosure provides for a composition, comprising a chemical having the following formula:

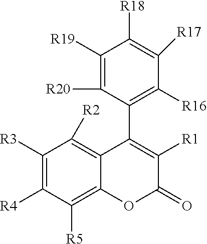

wherein R1 is an alkenyl group or an alkenyl group terminated by an acetoxy (OAc) group or a phenyl group; and wherein R2-R5, and R16-R20 are each independently selected from hydrogen, a halogen group, —OH, —CN, —C═N—S(O)(alkyl group), alkynyl group, or —O-alkyl.

### BRIEF DESCRIPTION OF THE DRAWINGS

The present disclosure may be better understood with reference to the following figures.

FIG. 1 illustrates Schemes 1A-1D. Scheme 1A illustrates a three component cine-ipso disubstitution. Scheme 1B illustrates cine-Substitution. Scheme 1C illustrates cine-ipso disubstitution via cycloaddition. Scheme 1D illustrates functional, structural, and three-dimensional complexity via cine-ipso disubstitution.

FIG. 2 illustrates Table 1, which shows select optimization reactions for three-component cine,ipso-disubstitution.

FIG. 3 illustrates Scheme 2 that illustrates a reaction scheme and various products.

FIG. 4 illustrates Scheme 3 that illustrates the re-examination of H—NO₂ elimination.

FIG. 5 illustrates Schemes 4A and 4B. Scheme 4A illustrates Point-to-axial chirality transfer depends on the energetics of rotamerization and elimination, while Scheme 4B illustrates Point-to-axial chirality transfer observed.

FIG. 6 illustrates Scheme 5 that shows functional group interconversion to structurally and functionally complex indole-coumarins.

FIG. 7 illustrates Scheme 6 that illustrates other aromatic nucleophiles (A) and electrophiles (B) yielding cine,ipso-disubstitution of nitrocoumarins.

FIG. 8 illustrates Schemes 7A and 7B. Scheme 7A shows cine,ipso-disubstitution with concomitant trans-lactonization, while Scheme 7B shows pKa dependent trans-lactonization.

### DETAILED DESCRIPTION

### Definitions

It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc. It is understood, unless otherwise stated, the various compounds include substituted compounds and/or groups (e.g., an R group) can be substituted.

It will be understood by those skilled in the art that the moieties substituted can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The terms “alkoxyl” or “alkoxyalkyl” as used herein refer to an alkyl-O— group wherein alkyl is as previously described. The term “alkoxyl” as used herein can refer to C₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

The term “alkyl” as used herein, means a monovalent, saturated hydrocarbon radical which may be a straight chain (i.e. linear) or a branched chain. An alkyl radical for use in the present disclosure generally comprises from about 1 to 20 carbon atoms, particularly from about 1 to 10, 1 to 8 or 1 to 7, more particularly about 1 to 6 carbon atoms, or 3 to 6, or 1 to 3, or 1 to 2. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-actyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the disclosure an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents as defined herein at positions that do not significantly interfere with the preparation of compounds of the disclosure and do not significantly reduce the efficacy of the compounds. In certain aspects of the disclosure, an alkyl radical is substituted with one to five substituents including halo, lower alkoxy, lower aliphatic, a substituted lower aliphatic, hydroxy, cyano, nitro, thio, amino, keto, aldehyde, ester, amide, substituted amino, carboxyl, sulfonyl, sulfuryl, sulfenyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, cycloaliphatic, substituted cycloaliphatic, or aryl (e.g., phenylmethyl benzyl)), heteroaryl (e.g., pyridyl), and heterocyclic (e.g., piperidinyl, morpholinyl). Substituents on an alkyl group may themselves be substituted.

The term “alkenyl” as used herein is a hydrocarbon group of from 2 to 24 carbon atoms or 2 to 8 carbon atoms or 2 to 6 carbon atoms with a structural formula containing at least one carbon-carbon double bond. Asymmetric structures such as (R1R2)C═C(R3R4) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

As used herein, “alkynyl” or “alkynyl group” refers to straight or branched chain hydrocarbon groups having 2 to 40, 2 to 20, 2 to 10, or 2 to 5 carbon atoms and at least one triple carbon to carbon bond, such as ethynyl. Reference to “alkynyl” or “alkynyl group” includes unsubstituted and substituted forms of the hydrocarbon moiety.

The Ar group is an aromatic system or group such as an aryl group. “Aryl”, as used herein, refers to C₅-C₂₀-membered aromatic, heterocyclic, fused aromatic, fused heterocyclic, biaromatic, or bihetereocyclic ring systems. In an aspect, “aryl”, can include 5-, 6-, 7-, 8-, 9-, and 10-membered single-ring aromatic groups that may include from zero to four heteroatoms, for example, functional groups that correspond to benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems (C₅-C₃₀) having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “carboxyl” as used herein, alone or in combination, refers to —C(O)OR— or —C(—O)OR, where R is hydrogen, alkyl, alkenyl, alkynyl, cycloalkyl, cycloalkenyl, amino, thiol, aryl, heteroaryl, thioalkyl, thioaryl, thioalkoxy, a heteroaryl, or a heterocyclic, which may optionally be substituted. In aspects of the disclosure, the carboxyl groups are in an esterified form and may contain as an esterifying group lower alkyl group. In particular aspects of the disclosure, —C(O)OR provides an ester or an amino acid derivative. An esterified form is also particularly referred to herein as a “carboxylic ester”. In aspects of the disclosure a “carboxyl” may be substituted, in particular substituted with allyl which is optionally substituted with one or more of amino, amine, halo, alkylamino, aryl, carboxyl, or a heterocyclic. Examples of carboxyl groups are methoxycarbonyl, butoxycarbonyl, tert.alkoxycarbonyl such as tert-butoxycarbonyl, arylmethyoxycarbonyl having one or two aryl radicals including without limitation phenyl optionally substituted by for example lower alkyl, lower alkoxy, hydroxyl, halo, and/or nitro, such as benzyloxycarbonyl, methoxybenzyloxycarbonyl, diphenylmethoxycarbonyl, 2-bromoethoxycarbonyl, 2-iodoethoxycarbonyltert.butylcarborlyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyi and dimethyltert.butylsilyl. In aspects of the disclosure, the carboxyl group may be an alkoxy carbonyl, in particular methoxy carbonyl, ethoxy carbonyl, isopropoxy carbonyl, t-butoxycarbonyl, t-pentyloxycarbonyl, sir heptyloxy carbonyl, especially methoxy carbonyl or ethoxy carbonyl.

### DISCUSSION

The present disclosure provides for polyfunctionalized biaryls having complex biaryl architectures, methods of making polyfunctionalized biaryls, and the like. The present disclosure provides for the cine-ipso-difunctionalization transformation of nitrocoumarins.

The reaction leverages the electrophilicity of nitrocoumarins, the nucleophilicity of nitronates, and the leaving group ability of nitrite (NO₂—) to yield complex, polyfunctionalized biaryls that often display stable axial chirality.

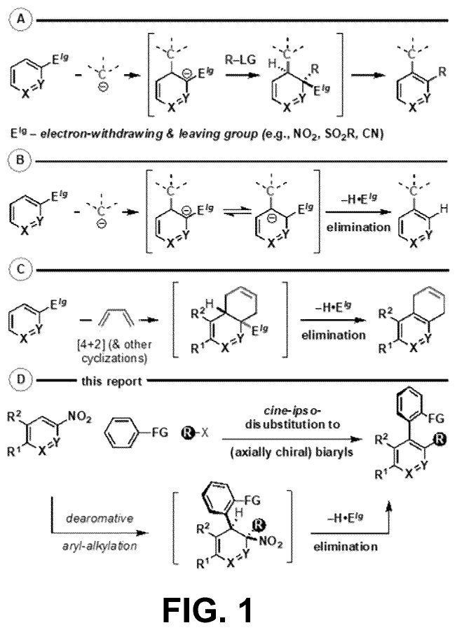

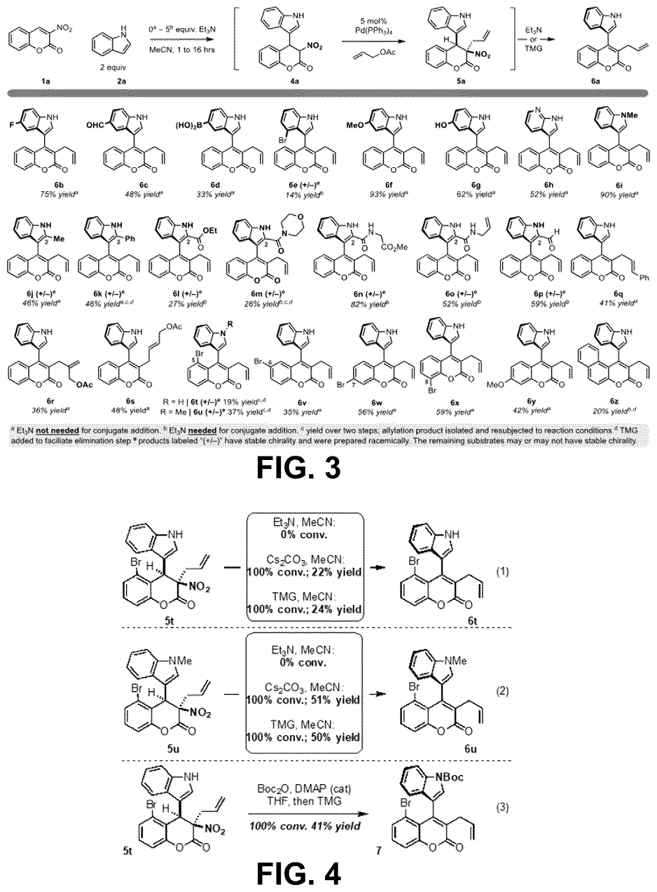

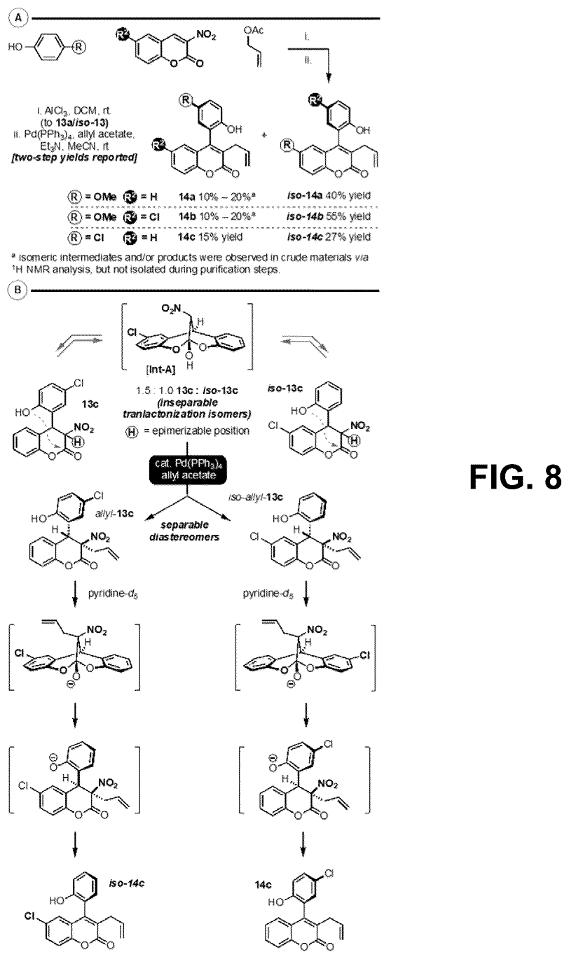

The present disclosure provides for transforming nucleophilic aromatic substitution from a monofunctionalization reaction to one capable of constructing two new bonds via a nitroarene dearomative conjugate addition, nitronate substitution, and eliminative aromatization strategy. The present disclosure describes the conceptualization of nitroarene cine,ipso-disubstitution via the three component coupling of nitrocoumarins, indoles, and allylic electrophiles (See Scheme 2 in Example 1). The method also resulted in a method that can rapidly populate indole-coumarin biaryl chemical space. Indole-coumarin biaryls are unique scaffolds due to the lack of methods capable of rapidly constructing diverse family members. The present disclosure allows access to these molecules enantioselectively through point-to-axial chirality transfer. The present disclosure also provides for the tunability and modularity of this strategy through functional group interconversion chemistry (See Scheme 5 in Example 1) and other coumarin disubstitutions (See Scheme 6 and 7 in Example 1): benozothiophene-allylation-, aryl-allylation-, indole-fluorination, indole-acrylation, and o-phenol-allylation of coumarins. Methods of making the polyfunctionalized biaryls of the present disclosure are provided in Example 1. In particular, the present disclosure provides for preparing the compounds described herein using the methods illustrated in FIGS. 1-8, where these methods can be adapted to substitute for each of the R groups described below and herein (e.g., R1-R20) based on these figures and the details provided in Example 1. For example, FIG. 3 and it associated discussion illustrates the method of making all of the compounds shown in FIG. 3. This can then be extended to other structures related to these.

In an aspect, the present disclosure provides for compositions with one or more chemicals described by the following formulae and those illustrated and described in Example 1:

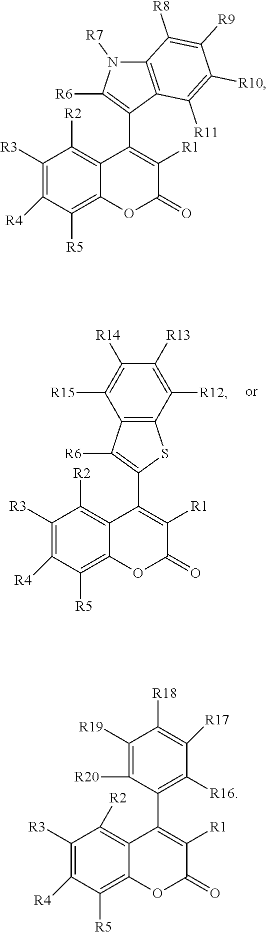

The present disclosure provides for any one of these structures independent of the others so that one of these can be present in the composition. Methods of making these polyfunctionalized biaryls are provided in Example 1.

In an aspect, R1 can be an alkenyl group or an alkenyl group terminated by an acetoxy (OAc) group or a phenyl group. For example, R1 can be a C2 to C6 or C2 to C4, or C3 alkenyl group. R1 can be an C2 to C6 or C2 to C4, or C3 alkenyl group with a terminal double bond such as —CH₂—CH═CH₂. Also, R1 can be C2 to C6 or C2 to C4, or C3 alkenyl group terminated by an acetoxy (—OAc) group or a phenyl group.

In an aspect, R2-R5, R6, R8, R9, and R11-R20 can each be independently selected from hydrogen, a halogen group, —OH, —CN, —C═N—S(O)(alkyl group) (e.g., C═N—S(O)(butyl)), alkynyl group (—C2-TMS or C3-TMS), or —O-alkyl (e.g., —O-methyl). For example, R2-R5, R6, R8, R9, and R11-R20 can each be independently selected from hydrogen or a halogen group (e.g., Br or F). In an aspect, R7 is H or an alkyl group (e.g., methyl group). In an aspect, R10 can be H, a halogen group, —CHO, —B(OH)₂, or —O(alkyl) (e.g., —O(methyl)). In an aspect, R10 the alkyl group can be a methyl group or ethyl group.

In an aspect, R6 can be selected from hydrogen, a halogen group, —OH, —CN, —C═N—S(O)(alkyl group) (e.g., C═N—S(O)(butyl)), alkynyl group (—C2-TMS or C3-TMS), —O-alkyl (e.g., —O-methyl), an alkyl group (e.g., methyl group), a phenyl group, —C(O)O-alkyl (e.g., —C(O)O-ethyl), —C(O)H, —C(O)R30 (e.g., R30 is a morpholine (bonded via the N group of morpholine) or another hetero-ring with at least two heteroatoms in the ring), —C(O)NH(alkenyl), or —COHNR31 (e.g., R31 is hydrogen, a halogen group, or an alkyl group (e.g., —CH₂—CH═CH₂)).

In an aspect, R6 can be selected from hydrogen, a halogen group, —OH, —CN, —C═N—S(O)(alkyl group) (e.g., C═N—S(O)(butyl)), alkynyl group (—C₂-TMS or C₃-TMS), or —O-alkyl (e.g., —O-methyl).

In another aspect, R6 can be hydrogen, a halogen group, an alkyl group (e.g., methyl group), a phenyl group, —C(O)O-alkyl (e.g., —C(O)O-ethyl), —C(O)H, —C(O)R30 (e.g., R30 is a morpholine (bonded via the N group of morpholine) or another hetero-ring with at least two heteroatoms in the ring), —C(O)NH(alkenyl), or —COHNR31 (e.g., R31 is hydrogen, a halogen group, or an alkyl group (e.g., —CH₂—CH═CH₂)).

In an aspect, R7 is H or an alkyl group (e.g., methyl group). In an aspect, R10 is H, a halogen group, —CHO, —B(OH)₂, or —O(alkyl) (e.g., —O(methyl)).

The present disclosure provides for the following compound:

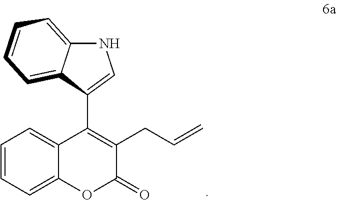

In addition, the present disclosure provides for the following compounds”

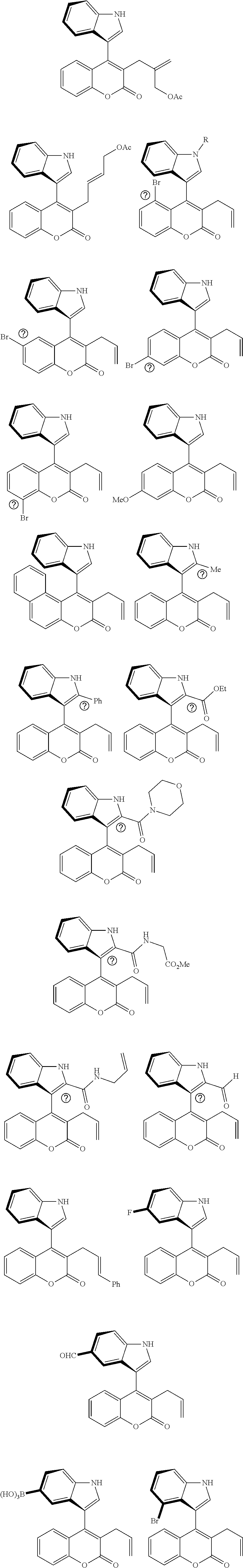

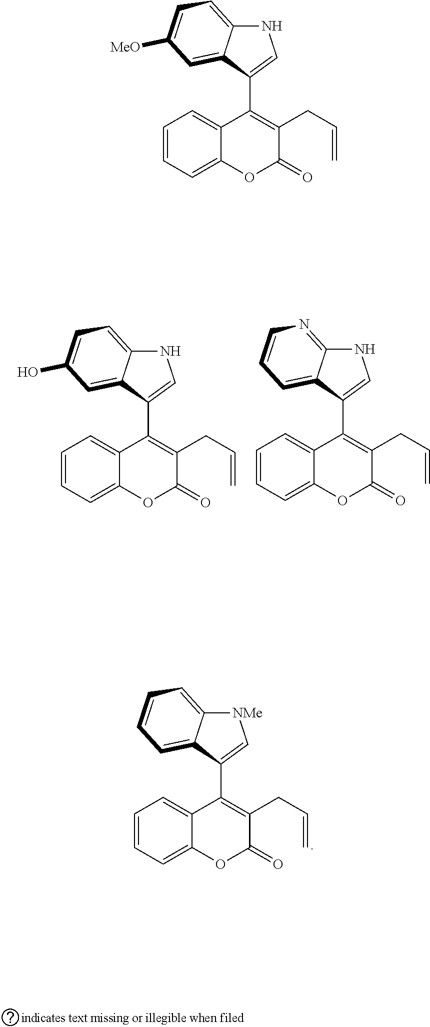

The present disclosure provides for:

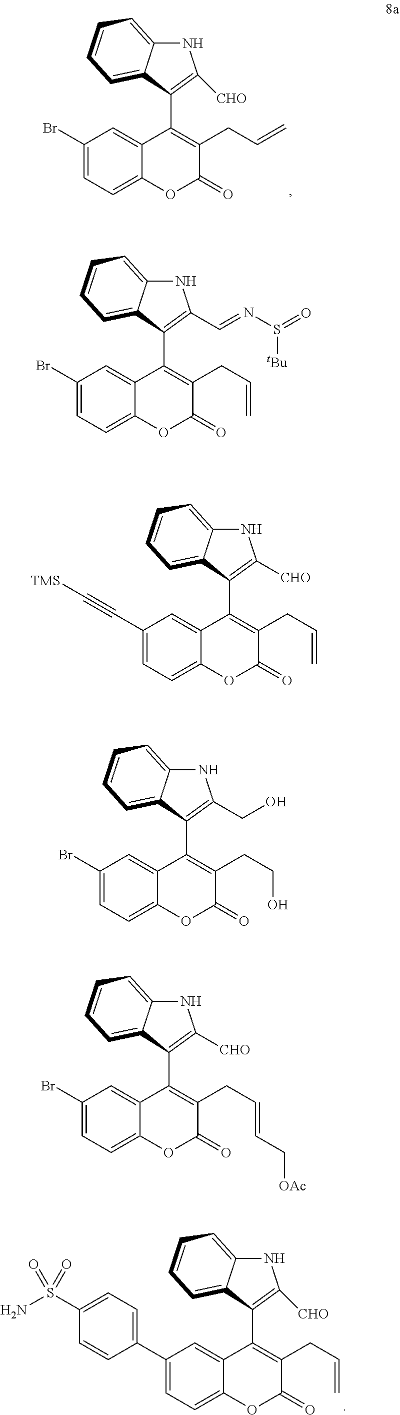

The present disclosure provides for:

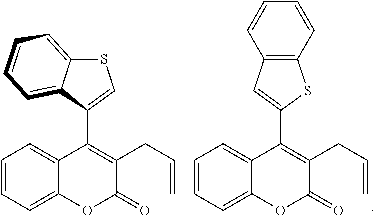

The present disclosure provides for:

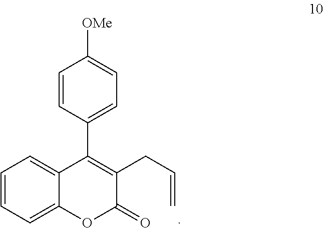 [SMILES: C=CCc1c(-c2ccc(OC)cc2)c2ccccc2oc1=O]

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example 1

Reported herein is the development of a cine,ipso-difunctionalization transformation of nitrocoumarins. The reaction leverages the electrophilicity of nitrocoumarins, the nucleophilicity of nitronates, and the leaving group ability of nitrite (NO₂) to yield complex, polyfunctionalized biaryls that often display stable axial chirality. This Example includes the development of the strategy, scope studies, representative applications toward complex biaryl architectures, and understanding related to point-to-axial chirality transfer in this system.

The linking of functional groups and (hetero)aromatics to (hetero)arene building blocks controllably and rapidly is critical considering the prominence of this pattern in pharmaceuticals. Modern strategies that achieve disubstitution include metal-catalyzed cross-coupling,¹ directed C—H functionalization,² the Catellani reaction,³ aryne chemistry,^{4,5} and photocatalysis transformations.⁶ Complimentary methods that achieve this goal and yield access to complex (functional, structural, and three-dimensional complexity) chemical space is essential to the drug discovery pipeline. We surmised that nitro(hetero)arenes (and potentially related arenes bearing an electron-withdrawing-leaving group (E_{Ig})) may serve as valuable starting materials for controlled, three-component (hetero)aromatic difunctionalization. Specifically, the nitro group could be harnessed three-fold (Scheme 1A): (1) as an activator for dearomative nucleophilic addition,⁷ (2) as a nitronate anion for diastereoselective alkylation,⁸⁻¹³ (3) and as a leaving group (pKa of nitrous acid (HNO2)=3.4).^{14,15} This reaction manifold, while simply outlined, is surprisingly understudied.¹⁶⁻¹⁹ If developed more broadly, this disubstitution strategy has the potential to transform nucleophilic aromatic substitution (classic-⁷, at hydrogen-²⁰⁻²², and vicarious-²³⁻²⁷ SNAr) from a mono-aromatic functionalization and interconversion strategy into one that yields two new C—C bonds from three, diversifiable components. Challenging this proposal is the monofunctionalization pathway that results in only cine-substitution (Scheme 1B).²⁸⁻³³ In terms of nitroarene (or related) cine,ipso-disubstitution, this is generally achieved through two component cycloaddition/rearomatization (Scheme 1C).³⁴⁻⁴³

In Example 1 a strategy a strategy is provided that harnesses the reactivity described in Scheme 1A (FIG. 1) and results in axially chiral biaryls (Scheme 1D) thus yielding products of high functional-, structural-, and often chiral-complexity.^{44,45} To begin our research toward this goal, we report the coupling of nitrocoumarins, indoles, and allyl acetates to yield axially chiral indole-coumarin biaryls bearing an alkene functional handle, explored the scope of the transformation, gained insight into point-to-axial chirality transfer⁴⁶⁻⁵⁰ through this strategy, and briefly examined scope beyond the initial methodology.

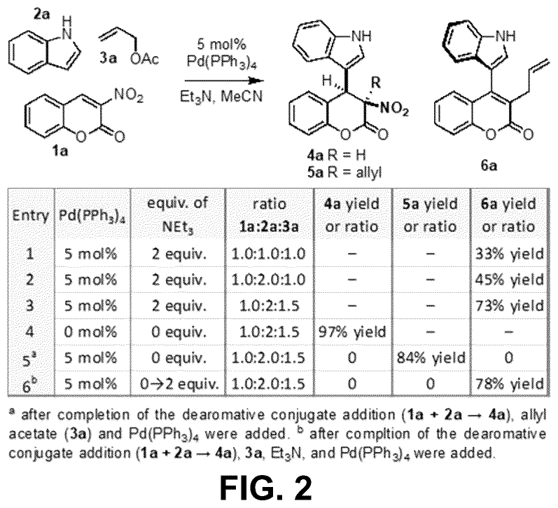

Our studies began with the examination of the three-component coupling of 3-nitrocoumarin 1a, indole 2a, and allyl acetate 3a to yield chiral indole-coumarin biaryl 6a (via 4a and 5a), a representative product of high value considering the ubiquity of indoles^{51,52} and coumarins^{53,54} in drug discovery. Select optimization results are summarized in Table 1 (FIG. 2). By simply mixing all components together in equimolar fashion in acetonitrile, using Et₃N (2 equiv.) as a base and Pd(PPh₃)₄ as a catalyst, a 33% isolated yield of 6a was observed (entry 1). Excess indole (entry 2) and indole/allyl acetate (entry 3) also improved the yield of the desired product. We next probed the individual steps of the transformation (entries 4-6). It was found that the dearomative conjugate addition reaction proceeds in the absence of triethylamine (entry 4)⁵⁵ and that the allylic alkylation does not need base (entry 5). As such, we ultimately uncovered a high yielding version of this transformation involving sequential addition of reagents/starting materials (entry 6).

Next examined was the scope of the transformation (Scheme 2, FIG. 3). First studied was indole substitution on the benzene ring (6b-6h). Electron withdrawing groups at the 5-position of indole were well tolerated (6b-6d) including often sensitive aldehydes (6c) and boronic acids (6d). Product 6e bearing a 4-bromo group yielded the desired product, but in modest yield. This was a steric problem associated with the conjugate addition step between nitro-coumarin 1a and 2e. Electron donating groups at the 5-position were also well tolerated (6f-6g). Notably, a free phenol was incorporated onto the biaryl scaffold (6g). We were also able to prepare product 6 h bearing a basic nitrogen from 7-azaindole. N-methylindole also was a competent coupling partner yielding product 6i. Indoles bearing various 2-functional groups were competent coupling partners, generally speaking. For example, methyl (6j), phenyl (6k), ester (61), amides (morpoline-(6m), glycinate-(6n), and allyl-(60) containing) and aldehyde (6p) functional groups were tolerated at the 2-position. That said, the ester-bearing product in this series gave a noticeably lower yield. It was observed that this particular indole (21) was a poor nucleophile for conjugate addition: it was unreactive under neutral conditions and yielded complex mixtures in the presence of base. Notably, all of the other 2-EWG-substituted indoles required the addition of Et₃N to promote the conjugate addition (e.g. 2m-2p→5m-5p; Scheme 2). With respect to the allyl acetate starting material, we were able to make products 6q-6s derived from cinnamyl acetate, and the acetates of 2-methylenepropane-1,3-diol and cis-butene-1,4-diol, respectively. The last examples in Scheme 2 show that substitution on the nitro-coumarin is also tolerated at many positions. For example, we prepared products containing a bromo-functional handle at the 5-8-positions of coumarin (6t-6x), examined electron-rich coumarins (6y) and prepared an indole-benzocoumarin (6z). Notably, the 5-substituted coumarins suffer from inefficient H—NO₂ elimination chemistry. For this elimination to occur, the respective intermediate 5t and 5u were first isolated (88% and 74% yield, respectively), and then the elimination step was re-optimized, resulting in 1,1,3,3-tetramethylguanidine (TMG) being identified as the superior base. Similarly, with other sterically congested substrates (e.g., 5m and 5z), TMG was also found to be the optimal base for elimination to 6m and 6z, respectively.

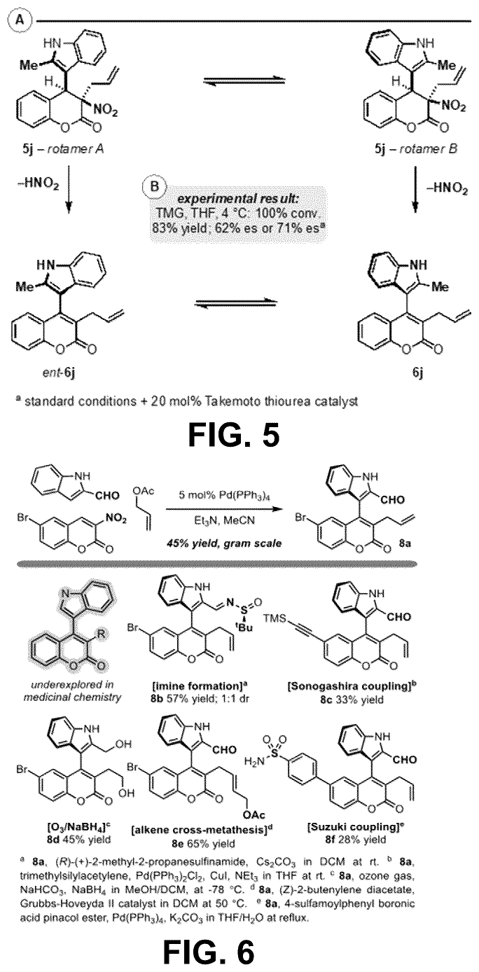

From the above outlined scopes studies, we observed that sterically demanding substrates often underwent inefficient Et₃N-promoted elimination. However, other bases such as TMG and Cs₂CO₃ could improve the outcome (Scheme 3, eq 1 (FIG. 5)). We also observed that the N-methyl indole product 6u could be prepared in higher yield compared to the analogous substrate with the free N—H (Scheme 3, eq 2). Considering these results, we hypothesized that the acidity of, or H-bonding to, the free indole N—H maybe negatively impacting the elimination chemistry. In agreement with this, we found that N-Boc-protection followed by elimination to 7 was comparably effective to the N-methyl indole outcome (Scheme 3, eq 3).

The products 6 from the three-component coupling clearly display axial chirality as the allylic signals are diastereotopic in the ¹H NMR. On this line, no signal coalescence was observed for 6a via VT-NMR up to 130° C. in DMSO-d6 and an enantioenriched sample of 6j was found to be conformationally stable. As such, we wished to examine the possibility of a point-to-axial chirality transfer in this type of system. We surmised that point to axial chirality transfer would be possible if the potential indole rotamers (5j-rotamer A and B, Scheme 4A) displayed significant energy differences (one conformer preferred over the other) or the rates at which the rotamers undergo HNO₂ elimination are significantly different (or both). Interestingly, it was found that 5j (er=>99:1; enantiomers separated via chiral supercritical fluid chromatography (SFC)) undergoes an elimination with 62-71% enantiospecificity (up to 85:15 er). While imperfect, this result is proof of concept for accessing unique axially chiral biaryls via an HNO₂ elimination method. Understanding more deeply the impact of rotamer-biasing and the elimination step will be key to this next development.

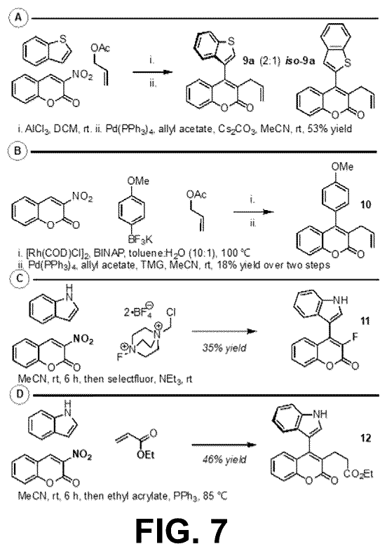

For the final studies, we wished to clarify and demonstrate the utility of indole-coumarin biaryls (Scheme 5, FIG. 6) and showcase that the transformation can produce unique biaryls beyond the initial scope (Scheme 6, FIG. 7). The chemistry developed herein yields complex and functionalized indole-coumarin biaryls. Notably, indoles and coumarins are both common to drugs and drug discovery efforts, though biological/pharmaceutical studies on their conjugates, indole-coumarin biaryls, are minimal. There are some reports of indole-coumarins in drug discovery campaigns, though studies and structural complexity about the system are limited.⁵⁶⁻⁶¹ It is possible that the bioactivity of indole-coumarins has remained understudied due to the lack of methods capable of constructing diverse and complex (structural, functional, and chiral complexity) analogs. As such, this work represents a direct and simple entry into novel and complex indole-coumarin biaryls with unknown bioactivity. Further, additional functional groups for diversification can be “dialed in” on the abundant starting materials. For example, we prepared 8a bearing four distinct functional groups (indole N—H, aldehyde, alkene, and Csp2-Br functional groups) from indole-2-carbaldehyde, allyl acetate, both of which are commercially available and the bromonitrocoumarin, prepared in one step from ethyl nitro-acetate and the requisite 5-bromosalicylaldehyde.⁶²In unoptimized studies, using standard conditions from the literature, 8a was successfully derivatized via imine formation (8b),⁶⁶ Sonogashira cross coupling (8c),⁶⁴ ozonolysis (with NaBH4 work-up),⁶⁵ cross metathesis (8b),⁶³ and Suzuki cross coupling (8f).⁶⁷ Thus, from commercial sources, products 8a-8f are prepared in two to three steps, showcasing that diverse, axially chiral indole-coumarins are now readily accessible.

In terms of the methodology, indole,allyl-cine,ipso-disubstitution of nitrocoumarins is a proof of concept study: In principle, coupling partners can be exchanged accessing an even greater diversity of chemical space. In this regard, we examined the coupling of a nucleophilic benzothiophene (Scheme 6A) and an arylBF₃K salt (Scheme 6B) as well as electrophilic SelectFluor™ (Scheme 6B) and ethyl acrylate (Scheme 6C) with nitro-coumarin. Somewhat surprising was that the benzothiophene did not react analogously to indole. Neutral conditions (benzothiophene+nitrocoumarin) did not promote the dearomative conjugate addition reaction. However 1 equiv. of AlCl₃ promoted this step as a 2:1 inseparable mixture of benzothiophene isomers 9a and iso-9a.⁶⁸ Utilizing tandem Rh(I) and Pd(0) catalysis, it was found that the aryl,allyl-coumarins could be prepared. The Rh(I) catalyst first promotes the dearomative conjugate addition. Then Pd(0) and TMG promotes the allylation and eliminative aromatization to 10. The yield for this two-step sequence is modest (18% over two steps), but is an exciting proof-of-concept result for coupling arylboronic acid derivaties, electrophiles, and nitrocoumarins. Regarding other electrophilic components, with little change in conditions (besides swapping allyl acetate/Pd(0) for SelectFluor™), we were able to prepare indole,fluoro-disubstituted coumarin 11 in 35% yield.⁶⁹ Regarding the coupling of indole and ethyl acrylate across nitrocoumarins, this required some additional optimization to yield the targeted product. The main hindrance was that the Michael addition of ethyl acrylate to the nitroacetate (post indole addition) did not progress in the presence of Et₃N base. We surmised that the nitroacetate anion was too stable to undergo conjugate addition. As such, we turned to triphenylphosphine, which has a unique mechanism for promoting Michael additions.⁷⁰ Indeed, the coupling of indole, ethyl acrylate and nitrocoumarin ultimately progressed in the presence of triphenylphosphine to yield indole-coumarins 12 bearing a pendant ester in 46% yield. Interestingly, it appears that PPh₃ promoted both the Michael addition and the elimination step as no additional base was needed. These results suggest that there are many ways to consider difunctionalizing nitrocoumarins via a dearomative conjugate addition, substitution, and eliminative aromatization mechanism.

One final methodological deviation that yielded interesting and promising results was the cine,ipso-disubstitution of nitrocoumarins with phenolic nucleophiles (Scheme 7). It was observed that treating nitrocoumarins and phenols with AlCl₃ in CH₂Cl₂ followed by allyl acetate, Pd(PPh₃)₄, and Et₃N (standard allylation/H—NO₂ elimination conditions) yielded unexpected isomeric products as the major products (iso-14) and the “originally anticipated” products 14 in minor amounts (Scheme 7A). In limited scope and optimization studies, the p-methoxylphenol nucleophile resulted in good yields of iso-14a (40% yield) and iso-14b (55% yield), considering that multiple C—C bonds are constructed. The minor products, 14a and 14b, could not be isolated cleanly by silica gel chromatography, but were observed during analysis of crude materials. When p-chlorophenol was used as the nucleophile through the sequence, a 27% yield of iso-14c was achieved along with isolable and characterizable minor product 14c. After careful experimentation and NMR analysis (including ¹H, ¹³C, gHMBC, nOe analyses; see Supporting Information for details), it was determined that the products 14/iso-14 are arising via two distinct trans-lactonizations as described in FIG. 7B. The dearomative Michael addition between nitrocoumarin and 4-chlorophenol yields 13c and iso-13c. Data supports that 13c with trans-stereochemistry is stable, but the cis-diastereomer undergoes goes trans-lactonization to iso-13c via the intermediate tetrahedral intermediate [Int-A]. Pd-catalyzed allylation of 13c/iso-13c yields separable regioisomers allyl-13c and iso-allyl-13c with a cis-relationship between the allyl and the phenolic group. It is proposed that the phenolic group has a directing effect on the allylation diastereoselectivity. Examination of the elimination step for both allyl-13c and iso-allyl-13c revealed that each individual substrate undergoes trans-lactonization prior to elimination yielding iso-14c and 14c, respectively. This is rationalized by the fact that the aromative elimination reaction will prefer a trans-relationship between the “H” and “NO₂”. Regarding the Pd-catalyzed allylation step, the reaction is chemoselective for the nitroacetate over the phenol nucleophile. Nitroacetates and phenols are both highly competent nucleophiles in Tsuji-Trost allylation chemistry⁷¹. As such, we suspect that the in situ generated carbonate base selectively deprotonates the more acidic nitroacetate, thus allowing for chemoselectivity to be achieved. As a final point of interest, products 14 clearly display stable axial chirality. By ¹H NMR and chiral HPLC analyses, observed are enantiotopic allylic signals and separation of atropisomers, respectively.

We have outlined a strategy for transforming nucleophilic aromatic substitution from a monofunctionalization reaction to one capable of constructing two new bonds via a nitroarene dearomative conjugate addition, nitronate substitution, and eliminative aromatization strategy. This work primarily focused on conceptualization of nitroarene cine,ipso-disubstitution via the three component coupling of nitrocoumarins, indoles, and allylic electrophiles (Scheme 2). Doing so also resulted in a method that can rapidly populate indole-coumarin biaryl chemical space. Indole-coumarin biaryls are nearly unexplored scaffolds due to the lack of methods capable of rapidly constructing diverse family members. It is also possible to access these molecules enantioselectively through point-to-axial chirality transfer, though improvements in efficiency and better understanding of stereoconrol will be necessary to develop enantioselective variants. We also showcase the tunability and modularity of this strategy through functional group interconversion chemistry (Scheme 5) and other coumarin disubstitutions (Scheme 6 and 7): benozothiophene-allylation-, aryl-allylation-, indole-fluorination, indole-acrylation, and o-phenol-allylation of coumarins.

### REFERENCES FOR EXAMPLE 1

(1) M. J. Buskes, M.-J. Blanco Molecules 2020, 25, 3493.
(2) S. Rej, Y. Ano, N. Chatani Chem. Rev. 2020, 120, 1788-1887.
(3) N. Della Ca', M. Fontana, E. Motti, M. Catellani Acc. Chem. Res. 2016, 49, 1389-1400.
(4) J.-A. Garcia-López, M. F. Greaney Chem. Soc. Rev. 2016, 45, 6766-6798.
(5) J. He, D. Qiu, Y. Li Acc. Chem. Res. 2020, 53, 508-519.
(6) P. Li, J. A. Terrett, J. R. Zbieg ACS Med. Chem. Lett. 2020, 11, 2120-2130.
(7) The SNAr Reactions: Mechanistic Aspects. In Modern Nucleophilic Aromatic Substitution; John Wiley & Sons, Ltd, 2013; pp 1-94.
(8) M. C. Dobish, J. N. Johnston, Org. Lett. 2010, 12, 5744-5747.
(9) R. S. Kim, L. V. Dinh-Nguyen, K. W. Shimkin, D. A. Watson Org. Lett. 2020, 22, 8106-8110.
(10) W.-O. Jung, B. K. Mai, B. J. Spinello, Z. J. Dubey, S. W. Kim, C. E. Stivala, J. R. Zbieg, P. Liu, M. J. Krische J. Am. Chem. Soc. 2021, 143, 9343-9349.
(11) C.-B. Ji, Y.-L. Liu, Z.-Y. Cao, Y.-Y. Zhang, J. Zhou Tetrahedron Lett. 2011, 52, 6118-6121.
(12) A. J. Grenning, J. A. Tunge Org. Lett. 2010, 12, 740-742.
(13) A. A. S. Gietter-Burch, V. Devannah, D. A. Watson Org. Lett. 2017, 19, 2957-2960.
(14) W. Raimondi, D. Dauzonne, T. Constantieux, D. Bonne, J. Rodriguez Eur. J. Org. Chem. 2012, 2012, 6119-6123.
(15) P. Liu, X. Bao, J.-V. Naubron, S. Chentouf, S. Humbel, N. Vanthuyne, M. Jean, L. Giordano, J. Rodriguez, D. Bonne J. Am. Chem. Soc. 2020, 142, 16199-16204.
(16) F. Hao, H. Asahara, N. Nishiwaki Tetrahedron 2017, 73, 1255-1264.
(17) R.-N. Guo, X.-F. Cai, L. Shi, Z.-P. Chen, Y.-G. Zhou Chem. Eur. J. 2014, 20, 8343-8346.
(18) P. Mampuys, T. D. Moseev, M. V. Varaksin, J. De Houwer, C. M. L. Vande Velde, O. N. Chupakhin, V. N. Charushin, B. U. W. Maes Org. Lett. 2019, 21, 2699-2703.
(19) T. Verhelst, S. Verbeeck, O. Ryabtsova, S. Depraetere, B. U. W. Maes Org. Lett. 2011, 13, 272-275.
(20) M. Mckosza Chem. Soc. Rev. 2010, 39, 2855-2868.
(21) M. Makosza, K. Wojciechowski Chem. Rev. 2004, 104, 2631-2666.
(22) O. N. Chupakhin, V. N. Charushin Tetrahedron Lett. 2016, 57, 2665-2672.
(23) D. Antoniak, B. Paluba, T. Basak, K. Blaziak, M. Barbasiewicz Chem. Eur. J. 2022, 28, e202201153.
(24) D. Antoniak, M. Barbasiewicz Org. Lett. 2022, 24, 516-519.
(25) J. Czaban-Jóźwiak, R. Loska, M. Makosza J. Org. Chem. 2016, 81, 11751-11757.
(26) V. V. Khutorianskyi, B. Klepetářová, P. Beier Org. Lett. 2019, 21, 5443-5446.
(27) J. Brzeskiewicz, R. Loska, M. Mckosza J. Org. Chem. 2018, 83, 8499-8508.
(28) J. W. Suwiński Arkivoc 2017, 2017, 402-435.
(29) W.-C. Yuan, X.-J. Zhou, J.-Q. Zhao, Y.-Z. Chen, Y. You, Z.-H. Wang Org. Lett. 2020, 22, 7088-7093.
(30) S. Bfa\hat{z}ej, A. Kwast, M. Mckosza Tetrahedron Lett. 2004, 45, 3193-3195.
(31) F. Berger, E. M. Alvarez, N. Frank, K. Bohdan, M. Kondratiuk, L. Torkowski, P. S. Engl, J. Barletta, T. Ritter Org. Lett. 2020, 22, 5671-5674.
(32) N. S. Zimnitskiy, A. Y. Barkov, I. A. Kochnev, I. B. Kutyashev, V. Y. Korotaev, V. Y. Sosnovskikh New J. Chem. 2022, 46, 16047-16057.
(33) A. N. Komogortsev, B. V. Lichitsky, V. G. Melekhina, M. E. Minyaev Tetrahedron Lett. 2021, 68, 152939.
(34) R. Rkein, A. Bigot, L. Birbaum, M. Manneveau, M. D. Paolis, J. Legros, I. Chataigner Chem. Commun. 2021, 57, 27-44.
(35) W.-C. Yuan, X.-M. Chen, J.-Q. Zhao, Y.-P. Zhang, Z.-H. Wang, Y. You Org. Lett. 2022, 24, 826-831.
(36) J.-R. Zhuo, B.-X. Quan, J.-Q. Zhao, M.-L. Zhang, Y.-Z. Chen, X.-M. Zhang, W.-C. Yuan Tetrahedron 2020, 76, 131115.
(37) Z. Jiang, X. Liu, H. Zhang, J.-P. Tan, X. Ren, G. Gao, T. Wang Adv. Synth. Catal. 2021, 363, 3115-3120.
(38) H. Huang, Q.-Z. Li, Y.-Q. Liu, H.-J. Leng, P. Xiang, Q.-S. Dai, X.-H. He, W. Huang, J.-L. Li Org. Chem. Front. 2020, 7, 3862-3867.
(39) D. D. Della Rosa, J. P. Sanchez, M. N. Kneeteman, P. M. E. Mancini Tetrahedron Lett. 2011, 52, 2316-2319.
(40) X.-J. Zhou, J.-Q. Zhao, X.-M. Chen, J.-R. Zhuo, Y.-P. Zhang, Y.-Z. Chen, X.-M. Zhang, X.-Y. Xu, W.-C. Yuan J. Org. Chem. 2019, 84, 4381-4391.
(41) D. Cao, G. Chen, D. Chen, Z. Xia, Z. Li, Y. Wang, Y.; D. Xu, D.; J. Yang ACS Omega 2021, 6,16969-16979.
(42) D. Cao, D. Chen, G. Chen, H. Mo, Z. Xia, K. Li, J. Yang Asian J. Org. Chem. 2020, 9, 1610-1616.
(43) J.-Q. Zhao, X.-J. Zhou, Y.-Z. Chen, X.-Y. Xu, X.-M. Zhang, W.-C. Yuan Adv. Synth. Catal. 2018, 360, 2482-2487.
(44) S. T. Toenjes, J. L. Gustafson Future Med. Chem. 2018, 10, 409-422.
(45) S. R. LaPlante, L. D. Fader, K. R. Fandrick, D. R. Fandrick, O. Hucke, R. Kemper, S. P. F. Miller, P. J. Edwards J. Med. Chem. 2011, 54, 7005-7022.
(46) S.-C. Zheng, Q. Wang, J. Zhu, Angew. Chem. 2019, 131, 9313-9317.
(47) F. Guo, L. C. Konkol, R. J. Thomson J. Am. Chem. Soc. 2011, 133,18-20.
(48) T. Hattori, M. Date, K. Sakurai, N. Morohashi, H. Kosugi, S. Miyano Tetrahedron Lett. 2001, 42, 8035-8038.
(49) W.-L. Xu, W.-M. Zhao, R.-X. Zhang, J. Chen, L. Zhou Chem. Sci. 2021, 12,14920-14926.
(50) T. T. Nguyen Org. Biomol. Chem. 2019, 17, 6952-6963.
(51) E. Vitaku, D. T. Smith, J. T. Njardarson J. Med. Chem. 2014, 57, 10257-10274.
(52) N. Chadha, O. Silakari Eur. J. Med. Chem. 2017, 134, 159-184.
(53) K. Kasperkiewicz, M. B. Ponczek, J. Owczarek, P. Guga, E. Budzisz Molecules 2020, 25, 1465.
(54) K. A. Scott, P. B. Cox, J. T. Njardarson J. Med. Chem. 2022, 65, 7044-7072.
(55) M.-C. Ye, Y.-Y. Yang, Y. Tang, X.-L. Sun, Z. Ma, W.-M. Qin Synlett 2006, 2006, 1240-1244.
(56) J. Song, Y.-F. Guan, W.-B. Liu, C.-H. Song, X.-Y. Tian, T. Zhu, X.-J. Fu, Y.-Q. Qi, S.-Y. Zhang Eur. J. Med. Chem. 2022, 238, 114467.
(57) S. Kumar, S. Mehndiratta, K. Nepali, M. K. Gupta, S. Koul, P. R. Sharma, A. K. Saxena, K. L. Dhar Org. Med. Chem. Lett. 2013, 3, 3.
(58) T. Guo, Y. Liu, Y.-H. Zhao, P.-K. Zhang, S.-L. Han, H.-M. Liu Tetrahedron Lett. 2016, 57, 4629-4632.
(59) P. R. Kamath, D. Sunil, A. A. Ajees, K. S. R. Pai, S. Das Bioorg. Chem. 2015, 63, 101-109.
(60) J.-S. Lan, L.-F. Pan, S.-S. Xie, X.-B. Wang, L.-Y. Kong MedChemComm 2015, 6, 592-600.
(61) G. Delogu, C. Picciau, G. Ferino, E. Quezada, G. Podda, E. Uriarte, D. Vina Eur. J. Med. Chem. 2011, 46, 1147-1152.
(62) R. K. Sharma, Priyanka, D. Katiyar Chem. Mon. 2016, 147, 2157-2161.
(63) A. K. Chatterjee, T.-L. Choi, D. P. Sanders, R. H. Grubbs J. Am. Chem. Soc. 2003, 125, 11360-11370.
(64) M. Schilz, H. Plenio J. Org. Chem. 2012, 77, 2798-2807.
(65) S. G. Van Ornum, R. M. Champeau, R. Pariza Chem. Rev. 2006, 106, 2990-3001.
(66) O. I. Afanasyev, E. Kuchuk, D. L. Usanov, D. Chusov Chem. Rev. 2019, 119, 11857-11911.
(67) S. E. Hooshmand, B. Heidari, R. Sedghi, R. S. Varma Green Chem. 2019, 21, 381-405.
(68) Z. Tu, B. Rama Raju, T.-R. Liou, V. Kavala, C.-W. Kuo, Y. Jang, Y.-H. Shih, C.-C. Wang, C.-F. Yao Tetrahedron 2009, 65, 2436-2442.
(69) J. Kwiatkowski, Y. Lu Org. Biomol. Chem. 2015, 13, 2350-2359.
(70) C. Gimbert, M. Lumbierres, C. Marchi, M. Moreno-Manas, R. M. Sebastien, A. Vallribera Tetrahedron 2005, 61, 8598-8605.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1 percent to about 5 percent” should be interpreted to include not only the explicitly recited concentration of about 0.1 weight percent to about 5 weight percent but also include individual concentrations (e.g., 1 percent, 2 percent, 3 percent, and 4 percent) and the sub-ranges (e.g., 0.5 percent, 1.1 percent, 2.2 percent, 3.3 percent, and 4.4 percent) within the indicated range. The term “about” can include traditional rounding according to significant figures of the numerical value. In addition, the phrase “about ‘x’ to ‘y’” includes “about ‘x’ to about ‘y’”.

The term “providing”, such as for “providing an article” and the like, when recited in the claims, is not intended to require any particular delivery or receipt of the provided item. Rather, the term “providing” is merely used to recite items that will be referred to in subsequent elements of the claim(s), for purposes of clarity and ease of readability.

Many variations and modifications may be made to the above-described aspects. All such modifications and variations are intended to be included herein within the scope of this disclosure and protected by the following claims.

1. A composition, comprising a chemical having the following formula: 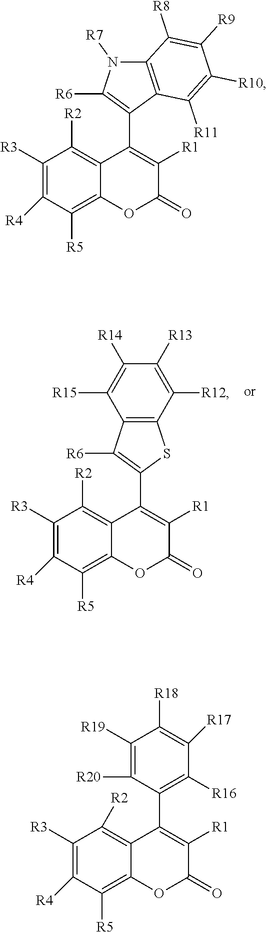
wherein R1 is an alkenyl group or an alkenyl group terminated by an acetoxy (OAc) group or a phenyl group;
wherein R2-R5, R8, R9, and R11-R20 are each independently selected from hydrogen, a halogen group, —OH, —CN, —C═N—S(O)(alkyl group), alkynyl group, or —O-alkyl;
wherein R6 is selected from hydrogen, a halogen group, —OH, —CN, —C═N—S(O)(alkyl group), —O-alkyl, an alkyl group, a phenyl group, —C(O)O-alkyl, —C(O)H, —C(O)R30, —C(O)NH(alkenyl), or —COHNR31, and wherein R30 is a morpholine group and wherein R31 is hydrogen, a halogen group, or an alkyl group;
wherein R7 is H or an alkyl group; and
wherein R10 is H, a halogen group, —CHO, —B(OH)₂, or —O(alkyl).

2. The composition of claim 1, wherein R1 is an alkenyl group.

3. The composition of claim 2, wherein the alkenyl group is —CH₂—CH═CH₂.

4. The composition of claim 1, wherein chemical is

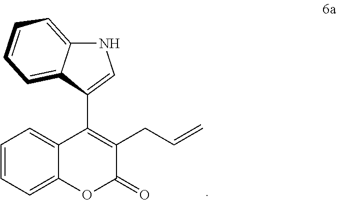

5. The composition of claim 1, wherein the chemical is selected from:

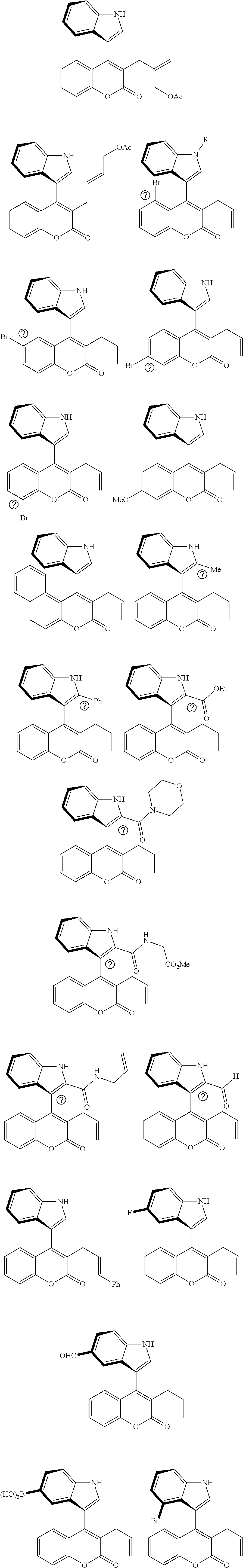

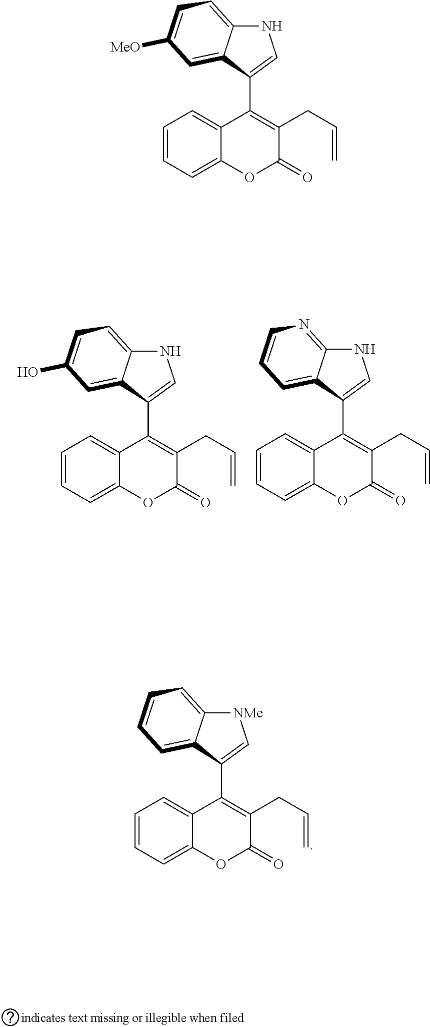

6. The composition of claim 1, wherein chemical is selected from:

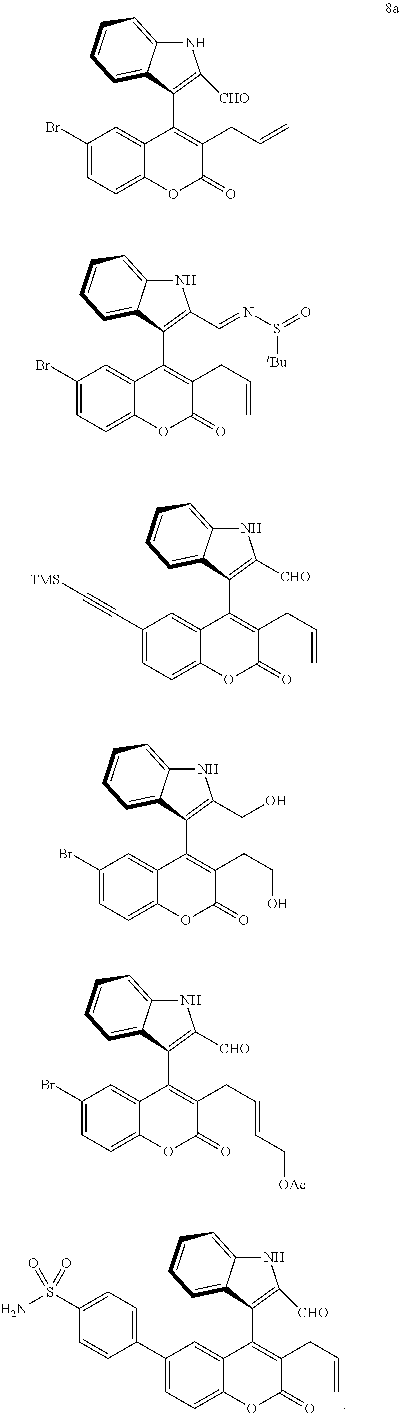

7. The composition of claim 1, wherein the chemical is selected from:

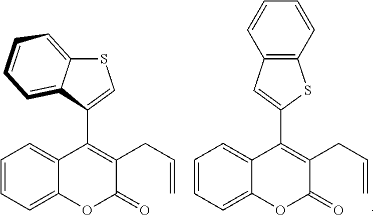

8. The composition of claim 1, wherein the chemical is

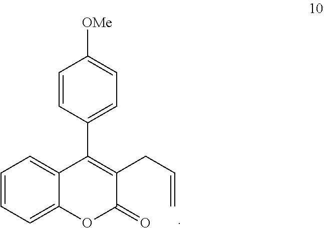 [SMILES: C=CCc1c(-c2ccc(OC)cc2)c2ccccc2oc1=O]

9. The composition of claim 1, wherein R6 is selected from hydrogen, a halogen group, —OH, —CN, —C═N—S(O)(alkyl group), alkynyl group, or —O-alkyl.

10. The composition of claim 1, wherein R6 is hydrogen, a halogen group, an alkyl group, a phenyl group, —C(O)O-alkyl, —C(O)H, —C(O)R30, —C(O)NH(alkenyl), or —COHNR31, and wherein R30 is a morpholine group and wherein R31 is hydrogen, a halogen group, or an alkyl group.

11. A composition, comprising a chemical having the following formula: 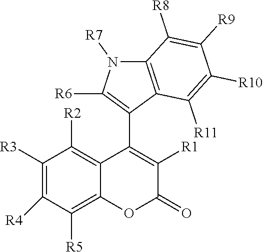
wherein R1 is an alkenyl group or an alkenyl group terminated by an acetoxy (OAc) group or a phenyl group;
wherein R2-R5, R8, R9, and R11 are each independently selected from hydrogen, a halogen group, —OH, —CN, —C═N—S(O)(alkyl group), alkynyl group, or —O-alkyl;
wherein R6 is selected from hydrogen, a halogen group, —OH, —CN, —C═N—S(O)(alkyl group), —O-alkyl, an alkyl group, a phenyl group, —C(O)O-alkyl, —C(O)H, —C(O)R30, —C(O)NH(alkenyl), or —COHNR31, and wherein R30 is a morpholine group and wherein R31 is hydrogen, a halogen group, or an alkyl group;
wherein R7 is H or an alkyl group; and
wherein R10 is H, a halogen group, —CHO, —B(OH)₂, or —O(alkyl).

12. A composition, comprising a chemical having the following formula: 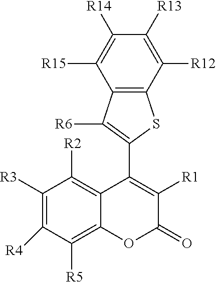
wherein R1 is an alkenyl group or an alkenyl group terminated by an acetoxy (OAc) group or a phenyl group;
wherein R2-R5, and R12-R15 are each independently selected from hydrogen, a halogen group, —OH, —CN, —C═N—S(O)(alkyl group), alkynyl group, or —O-alkyl;
wherein R6 is selected from hydrogen, a halogen group, —OH, —CN, —C═N—S(O)(alkyl group), —O-alkyl, an alkyl group, a phenyl group, —C(O)O-alkyl, —C(O)H, —C(O)R30, —C(O)NH(alkenyl), or —COHNR31, and wherein R30 is a morpholine group and wherein R31 is hydrogen, a halogen group, or an alkyl group;
wherein R7 is H or an alkyl group.

13. A composition, comprising a chemical having the following formula: 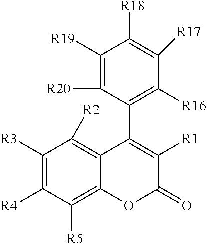
wherein R1 is an alkenyl group or an alkenyl group terminated by an acetoxy (OAc) group or a phenyl group; and
wherein R2-R5, and R16-R20 are each independently selected from hydrogen, a halogen group, —OH, —CN, —C═N—S(O)(alkyl group), alkynyl group, or —O-alkyl.
